(19) United States
(12) Patent Application Publication
(10) Pub. No.: US 2004/0030510 A1
(43) Pub. Date: Feb. 12, 2004

(54) Colour formulation method

(76) Inventors: Willem Frederik Van Biemen, Leiden (NL); Roelof Johannes Baptist Gottenbos, Leinderdorp (NL)

Correspondence Address: Joan M. McGillycuddy, Akzo Nobel Inc., Intellectual Property Department, 7 Livingstone Avenue, Dobbs Ferry, NY, 10522, US

(21) Appl. No.: 10/401,452

(22) Filed: Mar. 28, 2003

Related U.S. Application Data
(60) Provisional application No. 60/397,858, filed on Jul. 23, 2002.

(30) Foreign Application Priority Data
Mar. 28, 2002 (EP) 02076279.5

(51) Int. Cl. G01N 33/32; B01F 15/00; B65D 85/00

(52) U.S. Cl. 702/50; 427/8; 366/348; 356/402; 366/142; 702/25; 206/232

(57) ABSTRACT

Method for determining the colour formulation of a curable composition, e.g., a coating composition, which is produced by mixing one or more base compositions, e.g. base paints or toners, and which matches one or more pre-defined optical properties. The colour formulation is determined using batchwise determined base composition parameters, such as K and S values or parameters characterizing gloss, texture, metallic effect, etc. The base compositions are packed in containers comprising an information carrier, such as a bar code, making the batchwise determined parameters available.

This application claims the benefit of European Patent Application No. 02076279.5 filed on Mar. 28, 2002 and U.S. Provisional Application No. 60/397,858 filed on Jul. 23, 2002.

### FIELD OF THE INVENTION

The present invention relates to a colour formulation method for obtaining predefined optical properties in a curable composition produced by mixing a number of differently coloured, prefabricated base compositions. Colour formulation in this respect does not necessarily relate to obtaining a desired end product colour only, but may also involve other optical properties, such as metallic effect, gloss, structure, texture, etc.

### BACKGROUND OF THE INVENTION

In the paint industry, such systems are used to mix paint colours on the basis of a limited set of base paints, generally referred to as toners. Such a prior art paint colour mixing system is for example disclosed by U.S. Pat. No. 5,023,814, in which the base paints used are standardized compositions. The standardized base paints are distributed to for example points of sale or local production units, where they are used for mixing final paints according to standard formulations. This requires strict control of the base paint compositions. If a production batch of base paint deviates even slightly from the standardized base paint formulation, the batch cannot be used and needs to be adjusted or, if adjustment is too expensive or impossible, rejected in its entirety. Colour mixing using base compositions is also practiced in other fields of industry, e.g., in the field of inks, plastics or dyes for colouring fabrics or textiles.

### SUMMARY OF THE INVENTION

The object of the invention is to eliminate the need for adjustment or rejection of base composition batches and to eliminate the effects of base composition batch variations on the quality of the resulting paint formulation.

### BRIEF DESCRIPTION OF THE DRAWINGS

FIG. 1 Is a flow diagram of a method according to the invention;

FIG. 2 Is a diagram of the contents of Database II of FIG. 1.

### DETAILED DESCRIPTION OF THE INVENTION

The object of the invention is achieved by a colour formulation method wherein for at least one of the used base compositions the colorimetric data used for formulation of the final product are determined per batch of base composition. Instead of using data relating to standardized base compositions, use is made of data determined per batch. No corrections of the base compositions are needed anymore and batches are no longer rejected for not complying with pre-defined standards. In this respect a batch is to be defined as a quantity of base composition having the same calorimetric values, e.g., a supply of base paint of a certain composition. This can be for example be a single production batch or a blend of two or more production batches.

EP-A 0 131 414 discloses a method for paint production including the step of labeling base paint containers with the data of the colorants used, such as pigments. After selecting a pre-determined formulation for a final paint, the base paint is dosed as a function of the colorant content of the base paint. This publication does not disclose the use of calorimetric data of the base paint itself for determining a new formulation.

Preferably, use is made of colour mixing equipment controlled by a data processing device with access to a database of all base paints in active stock (the base paints readily available for colour formulation) and to a database of batchwise determined base paints parameters. Optionally, these databases may form a single, integrated database. Alternatively, the databases may be separate databases.

The data processing device can for example be a personal computer, a minicomputer or mainframe or any other suitable computer, which may optionally form part of a public or private computer network, such as an intranet or an extranet.

The location where the base paints are actually mixed to form a final paint giving the intended film properties may optionally be the same location as the one where the base paints are produced. However, in general these locations are separate, the formulation location typically being, for instance, a point of sale, a car repair body shop, a shipyard, an aircraft hangar, etc.

The database of base compositions in stock should be updated before a new base composition is used in a colour formulation location. This can for instance be done by manual input or by scanning labels or codes, such as bar codes. When the base composition of a certain formulation is exhausted, the database should be updated again. This can also be done by manual input, or alternatively the data processing device can register every amount used by the colour mixing equipment and remove a base composition from the database of base compositions in stock as soon as a certain amount of the base composition in question has been used. If the database of base compositions in stock also includes data concerning the amounts on stock, this data can be constantly or regularly updated by the data processing device.

The optical properties to be obtained using the method according to the present invention can for instance be colour (e.g., under different conditions of illumination), texture, gloss, and the like or combinations thereof.

If the colour is to be matched, the base composition parameters to be determined batchwise can for example be the K and S values according to the Kubelka Munk model (P. Kubelka and F. Munk, “Ein Beitrag zur Optik der Farbanstriche,” Z. tech. Physik., Ed. 12, page 593, 1931). In this system, the K parameter stands for the absorption factor. The S parameter is the scattering factor. Other models for determining the K and S values can also be used, for instance when metallic effects have to be taken into account. A suitable example of such a model is the Mulltiflux model as described in the article “Multiple Scattering Calculations for Technology” by P. S. Mudgett and L. W. Richards, Appl. Opt. (1971).

The K and S values of a base paint batch are determined by measuring base paint samples. The samples can be uncured, wet samples of the base paint, or they can be cured coating films. The reflection of the samples is measured at different wavelengths of visual light at regular intervals using a spectrophotometer, optionally at more than one angle.

Suitable spectrophotometers are for example the Automatchic® system, available from Akzo Nobel Coatings in Sassenheim, the Netherlands, the MacBeth Colour-Eye® 3000, the BYK-Gardner® 9300 handy-spec spectrophotometer, or the X-Rite® MA-68 II spectrophotometer.

A suitable way of characterizing gloss is for instance defined in International Standard ISO 2813 or US Standard ASTM E-340. Texture can for instance be characterized by parameterizing the particle shape and/or particle size distribution of the effect pigments (aluminum flakes, mica platelets, etc.) used in the base paint composition.

According to the present invention, batch related base composition parameters are used for formulating a final product composition in the same way as the K and S values of standardized base compositions have hitherto been used in prior art systems.

The final colour formulation can for example be determined by calculation or by data bank searching. Data bank searching requires the input of a product identifier, for example a car make with type designation and year of manufacture of a car model and/or measured colour data. In the databank, colour formulations are searched for which have, within pre-defined tolerances, substantially the same optical properties as the properties associated with the input data. These colour formulations are based on parameters relating to previous batches. The data of the previous batches is subsequently compared with the data of the base compositions presently in active stock. On the basis of this comparison, the formulation is modified in order to obtain an end product paint with the intended optical properties using the base compositions available for use.

In order to make the required calorimetric data available, the base compositions can be provided in containers with bar codes listing the colorimetric base paint data. Alternatively, the base composition containers are provided with one or more microchips storing colorimetric base composition data. An example of a suitable microchip based system is disclosed in EP-A 0 131 414.

Instead of labelling the base compositions with the parameterized batchwise determined information, the base composition containers can be provided with information identifying the corresponding batch in a form readable by a device having access to a database of batch-related data. This way, only minimal information needs to be stored on the container itself. The database can be consulted by an on-line connection via a communication network, e.g., an intranet or extranet construction or via the Internet or any other suitable computer network.

The method according to the invention can for instance be used in the production of do-it-yourself paints or professional paints, industrial paints, including powder coatings, coil coatings, aerospace paints, automotive paints or repair paints, e.g., in car refinishing. The term “base paint” also covers pigment pastes, toners, etc. Further, the method according to the invention can also be used in the field of inks, plastics or dyes for colouring fabrics or textiles.

The invention is further described and illustrated by the accompanying drawings. In the drawings:

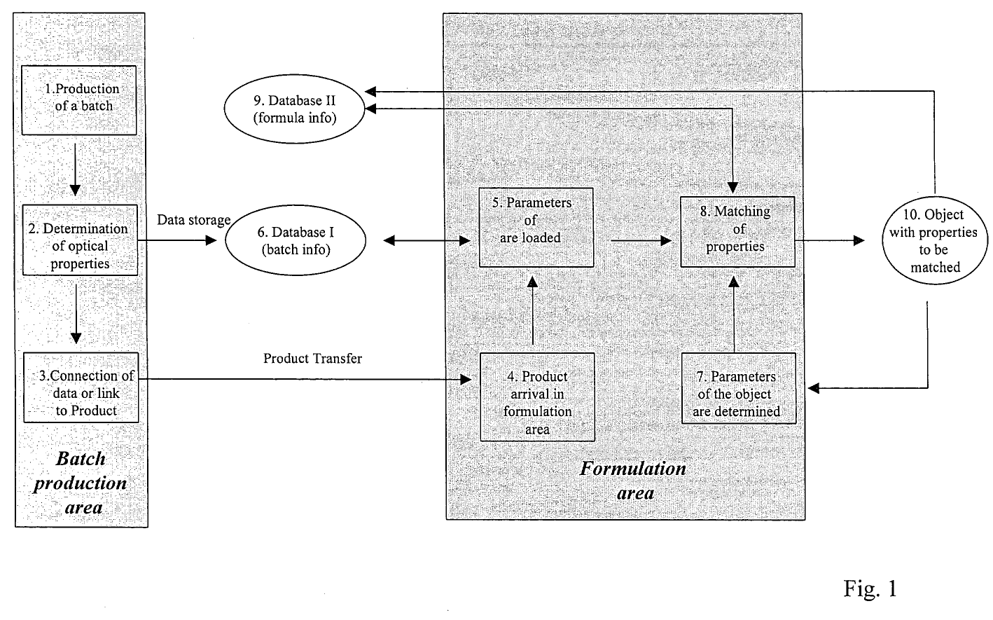

FIG. 1 shows a flow diagram of a method according to the invention;

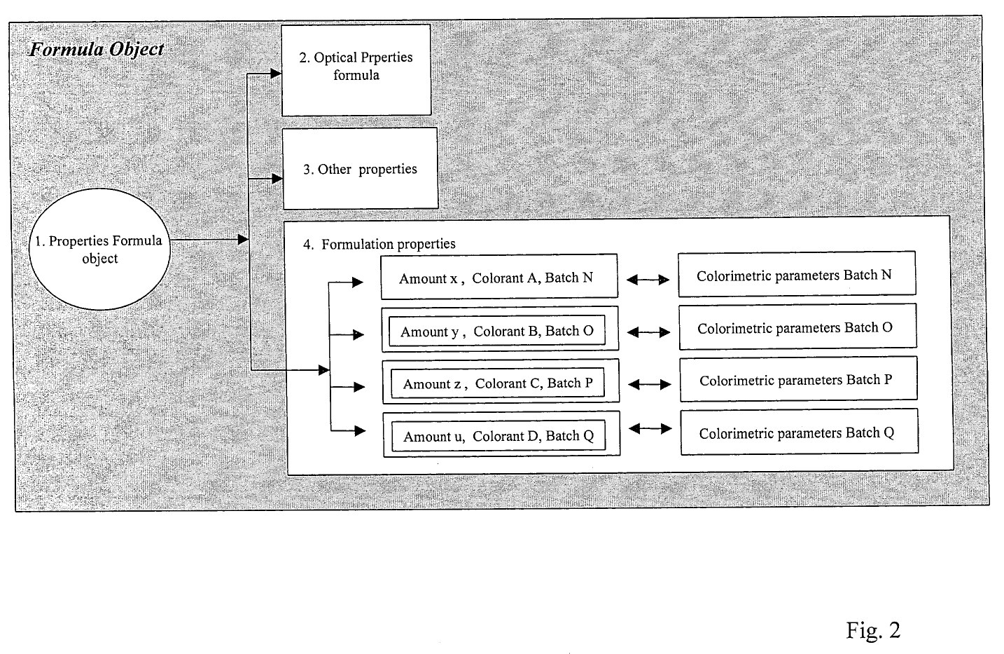

FIG. 2 shows the contents of Database II of FIG. 1.

In FIG. 1, an embodiment of the process according to the present invention is shown in a flow diagram. In a paint production area, batches of base paints are produced (box 1).

A set of a limited number of these base paints is used to mix a much broader range of colours and other optical paint film properties, such as metallic, pearlescent or dichroic effect, gloss, texture, etc.

In step 2, the optical properties of each batch of base paint are determined. This data is stored in Database 1 (Box 6).

Subsequently, the base paint is packaged in containers. The containers are provided with information identifying the batch, for instance by a bar code.

The product is shipped to a formulation area, for instance a car repair body shop. After delivery of the base paints, the information identifying the base paint batch is read from the containers. An entry into Database I is present in the formulation area, e.g., by means of a communication network such as the Internet or an extranet. Using the batch-identifying information read from the base paint containers, the optical properties of the base paints in the formulation area are downloaded and stored in a local database.

In a car repair body shop, damaged parts of a car (box 10) are refinished with a paint which must optically match the original car colour under various conditions of illumination. To this end, the optical properties of the original car paint are measured (box 7). In box 8, the locally stored batch parameters are used to determine a paint formulation which can be mixed using the base paints present in the car repair body shop and which matches the original paint of the car to be repaired.

For determining the matching paint, use can be made of Database II (box 9), which contains data of previously determined formulations. If the original paint of the car to be repaired has been matched previously, e.g. for repair of a car of the same colour, the previously determined formulation can be read from Database II together with the batch-related base paint data used for that formulation.

Subsequently, the batch-related base paint data is compared with the corresponding data of the actually present base paints. If there are any differences, the previously determined formulation is adjusted to come to a matching formulation, which can be mixed using the actually present base paints.

Database II is shown in more detail in FIG. 2. The database contains a set of properties. A distinction is made between optical properties, such as colour, texture, metallic effect, etc., and other properties, such as car make, car production site, car model, year of manufacture, etc. Each set of properties is linked to a colour formulation defined by its colorant content. For each colour formulation the required amount of each colorant is given, including a link to the production batch of the colorant in question. In the Example of FIG. 2, the set of properties is linked to a colour formulation comprising an amount x of colorant A of batch N plus an amount y of colorant B of batch 0 plus an amount z of colorant C of batch P plus an amount u of colorant D of batch Q. The third part of the database includes batchwise specified colorimetric parameters linked to the colour formulations using the colorant in question. If a new batch of the colorant is to be used, the colorimetric parameters of the new batch are introduced into the database, resulting in an adjustment of the colour formulation.

1. A method for determining the colour formulation of a curable composition which is produced by mixing one or more base compositions and which matches one or more pre-defined optical properties, wherein this colour formulation is determined using batchwise determined base composition parameters.

2. The method according to claim 1, wherein the base compositions are packed in containers comprising an information carrier making the batchwise determined parameters available.

3. The method according to claim 2, wherein the information carrier is a label with bar codes comprising the batchwise determined parameters.

4. The method according to claim 2, wherein the information carrier is a microchip storing the batchwise determined parameters.

5. The method according to claim 1, wherein the batchwise determined parameters include K and S values, texture parameters, and/or gloss parameters.

6. The method according to claim 1, wherein use is made of colour mixing equipment controlled by a data processing device having access to a database of batchwise determined base paint parameters of base paints in stock.

7. The method according to claim 1, wherein the formulation of the end product composition is determined by calculation.

8. The method according to claim 1, wherein the formulation of the end product composition is determined by data bank searching.

9. The method according to claim 1, wherein the base compositions are base paints used for the production of a car repair coating composition.

10. A base composition container comprising an information carrier, such as a bar code or a microchip, making batchwise determined base composition parameters available for colour formulation purposes.
